(19) United States
(12) Patent Application Publication
(10) Pub. No.: US 2008/0175963 A1
(43) Pub. Date: Jul. 24, 2008

(54) Beverage Freshness Detecting and Indicating Systems

(75) Inventor: Randy D. Pope, Edinburg, IL (US)

Correspondence Address: BARNES & THORNBURG LLP, P.O. BOX 2786, CHICAGO, IL, 60690-2786, US

(73) Assignee: BUNN-O-MATIC CORPORATION, Springfield, IL (US)

(21) Appl. No.: 11/627,851

(22) Filed: Jan. 26, 2007

Related U.S. Application Data
(60) Provisional application No. 60/763,106, filed on Jan. 26, 2006.

(51) Int. Cl. G01N 33/14; A47J 31/00

(52) U.S. Cl. 426/231; 99/285

(57) ABSTRACT

A system, method and apparatus for detecting the level of acidity or pH of material in a beverage container. The system includes a sensor assembly including a pH sensor. Preferably, a controller is provided and receives a signal from the sensor assembly for controllably operating functions of the beverage container or dispenser based on the detection signal from the sensor assembly relating to material in the container. The controller may prevent further dispensing or may indicate a level of freshness based on baseline pH values or a range of values for the particular beverage contained.

### RELATED APPLICATION

This application claims priority under 35 U.S.C. § 119(e) to U.S. Provisional Application Ser. No. 60/763,106 filed Jan. 26, 2006, which is expressly incorporated by reference herein.

### BACKGROUND

This disclosure relates generally to a beverage container for storing a beverage, and more particularly to an apparatus, system and method relating to detecting the level of acidity or pH of the material in a beverage container.

The detection of acidity or pH of the material within the container, which may generally be a hopper of a dispensing apparatus, a coffee or tea pot, a decanter, carafe, a serving container or any other vessel in which a beverage is stored prior to consumption, is important for a number of reasons. Beverages, particularly brewed beverages such as teas and coffees and their associated drinks do not retain the same level of freshness or taste indefinitely. Often, after a beverage has been stored for a period of time, the “freshness” of the beverage may deteriorate to an undesirable or unacceptable level with respect to a consumer of the beverage, and a new beverage should be prepared. An indication of a low level of “freshness” of the material in the container warns the operator that a new beverage should be prepared, as the previous beverage has surpassed an acceptable taste level or “freshness.” Also, informing the operator of the level of freshness of the material in the container reduces the potential for serving inferior quality or otherwise unacceptable or undesirable beverages because the freshness of the beverage measured or determined, at least in part by pH, has exceeded a predetermined limit or range.

Freshly prepared beverages, such as coffees, have a certain pH or acidity associated with that beverage. Factors such as coffee bean origin, level of roasting, pH of brew water regional or other flavor preference profiles, and other characteristics of the brewing elements may determine or otherwise contribute to the level of pH for a “fresh” beverage. Over time, however, depending on several factors such as the beginning pH level, the “freshness” decays and the pH of the beverage becomes more or less acidic. For coffee-based beverages, the pH level generally becomes more acidic over time until it reaches an undesirable level. By way of example but not limitation, the pH of a freshly brewed coffee beverage may generally be approximately 5.5 pH (pH=7 is neutral) and may change to a pH of approximately 4.5 whereupon it would be associated with an undesirable beverage. The starting and ending pH values may differ from beverage to beverage depending on a variety of factors, including but not limited to roast level of coffee beans time duration during which the coffees is heated and pH level of the brew water and others not in this disclosure. The time to an undesirable may also vary due to at least some of the same factors.

The present disclosure relates to a system, a method and an apparatus which involves sensing the pH of a beverage in a container. The present disclosure employs a pH sensor assembly to detect the acidity level of material in the container. The container may be an opaque or a generally transparent structure. The sensor assembly is positionable at a desired level of the container, such as near or adjacent the bottom thereof, so that the condition of the coffee may be sensed even at low volumes.

pH sensor of known construction or of construction yet to be developed suitable for food contact or at least for sensing of food material pH and operable in a solution may be used. Generally, the sensor assembly generates a signal that may be transmitted to a controller by way of electrical wiring, optical fiber or wirelessly. The sensor assembly alone or in combination with the controller may be incorporated, by way of example but not limitation, into a lid assembly for the container, the associated housing of the container or brewer, or may be provided as a device separate from but associated with the container. Multiple sensor assemblies can be used in a multiple container machine to sense the acidity level in each of the corresponding containers. Multiple controllers may be incorporated in such a machine, however a single controller capable of receiving a signal from the separate sensor assemblies might be preferred in some situations.

The present disclosure provides a pH level sensor assembly for a beverage container. One embodiment of the beverage container according to the present disclosure includes a container, a sensor assembly, and a controller. The sensor assembly detects the pH level of material in the container. The controller receives a signal from the sensor assembly and may control the function of the container or may control a user interface display or other signaling device depending on the output of the sensor assembly. If the sensor assembly senses a predetermined pH level or range or a total change in pH reaching a pre-determined level, the controller may operate to restrict dispensing of beverage or may operate a warning display or alarm for the user. If the sensor does not sense a pH level or change in pH sufficient to meet pre-determined levels, the container may dispense beverage.

The present disclosure provides a method of monitoring beverage freshness. A beverage is dispensed into a beverage container including a pH sensor assembly. The method comprises the steps of preparing a beverage and retaining the beverage in a container, sensing the pH value of the beverage, comparing the sensed pH value against a pre-determined pH limit or range. The sensor pH may be used for notification, control, alarm, as well as other functions. In another method according to the present disclosure, the method comprises the steps of preparing a beverage, holding the beverage in a container, sensing the pH value of the beverage, comparing the sensed pH value against a pre-determined pH limit value and restricting the dispensing of beverage from the container.

Additional features will become apparent to those skilled in the art upon consideration of the following detailed description of drawings exemplifying the best mode as presently perceived.

### BRIEF DESCRIPTION OF THE DRAWINGS

The present disclosure will be described hereafter with reference to the attached drawings which are given as a non-limiting example only, in which:

FIG. 1 is a diagrammatic illustration of an embodiment of a freshness sensing system including a beverage container, pH sensor, controller and indicator;

FIG. 2 is a diagrammatic side view of one embodiment of a beverage container according to the present disclosure;

FIG. 3 is a diagrammatic top plan view of a beverage system having multiple beverage containers; and

FIG. 4 is a diagrammatic side view of another beverage container according to the present disclosure.

The exemplification set out herein illustrates embodiments of the disclosure that is not to be construed as limiting the scope of the disclosure in any manner.

### DETAILED DESCRIPTION

While the present disclosure may be susceptible to embodiment in different forms, there is shown in the drawings, and herein will be described in detail, embodiments with the understanding that the present description is to be considered an exemplification of the principles of the disclosure and is not intended to be exhaustive or to limit the disclosure to the details of construction and the arrangements of components set forth in the following description or illustrated in the drawings.

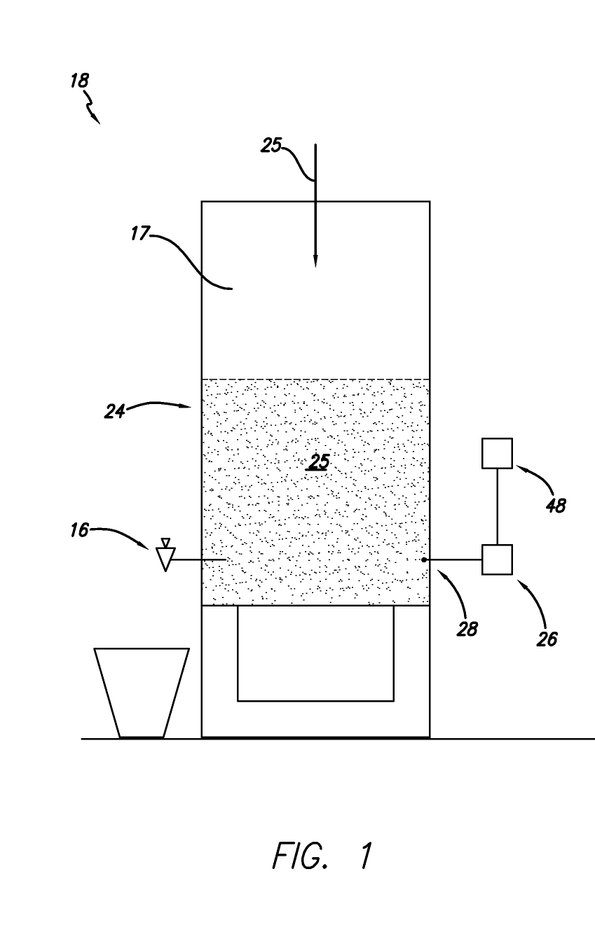

FIG. 1 shows a freshness detecting system 18 which includes a container 24, a pH sensor assembly 28, controller 26 and an indicator or other output 48. In this broad general embodiment beverage 25 or material is disposed into a cavity 17 of the container 24 over tension and subsequent dispensing therefrom. In one embodiment, beverage 25 is dispensed through a dispensing faucet or nozzle 16 associated with the container 24. The use of a nozzle is by no means limiting in the present disclosure as other forms of dispensing or controlled dispensing of beverage 25 from the container 24 is included in the present disclosure.

The pH sensor 28 may be positioned in the cavity 17 or otherwise associated with the container 24. pH sensor 28 will sense the pH of the beverage 25 retained in the container 24. A signal from the sensor is transmitted to the controller 26 by way of a wired, fiber optic, wireless or other connection to the controller. The controller 26 may be coupled to one or more devices including, as described herein below, valves or other control devices and, as shown in FIG. 1, a display, warning, indicia, auditory signal or other sensory device which will may indicate a condition of the beverage 25 retained within the container 24.

By way of example, if the desired beverage is brewed to a desired set of characteristics, it may have a starting or preferred pH of, approximately 5.5. The sensor 28 will sense the pH of the beverage 25 and continuously or periodically provide information to the controller 26. The controller 26 will monitor and, if desirable, act or provide signals in response to the pH level sent. For example, the controller 26 may be set to accept a pH in the range of 5.5-4.5. However, when the sensor indicates that the pH level has dropped to a level of 4.6, the controller 26 may take action by way of the display or other sensory device 48. Essentially, the controller 26 may provide a generally continuous signal to the display or sensory device 48 to indicate a real time or current condition as well as a periodic condition or “snapshot” of the pH of the beverage 25. The user or consumer may use this information for a variety of reasons. By way of example, but not limitation, a consumer of coffee may decide to chose one container 24 or another depending on the freshness level of the coffee retained therein. Alternatively, the sensory device 48 may be useful to the operator of a coffee seller such as a restaurant to indicate to the user the level of freshness of the coffee 25 retained in the container 24. With regard, the output from the sensory device 48 may be provided on a faucet or forward face of the container 24 or may be positioned away from the faucet side. It may be desirable to provide information towards the public or faucet side of the container 24 or may be desirable to not display it to the public side. In this regard, the information could be transmitted to a sensory device 48 at a location removed from the container 24 or even the facility in which it resides. In this regard, the signal may be transmitted to a master control signal or, for example, a service counter or register in a coffee selling location. This will provide information to the user or operator of the equipment as to the freshness levels. By way of example, but not limitation, in a C-store or convenience store setting, the information may be displayed at the front register. This may be useful to indicate to the operator that a fresh batch of coffee needs to be brewed to prevent undesirable tasting coffee from being sold.

In showing FIG. 1, the pH sensors positioned in the lower portion of the cavity 17 to sense the pH of beverage 25 even at lower volumes. The sensor 28 may be positioned in any desirable location including, but not limited to, the bottom of the container 24 as well as near the faucet 16 to detect the pH condition of the beverage. While an example of pH level starting at 5.5 and deteriorating to 4.5 has been provided, there is no intention of limiting the disclosure to this specific range. For example, there may be some configurations or coffee recipes in which the pH level of 4.5 is desirable but a pH level of 4.7 is undesirable. This may be possible as the result of the pH system being a logarithmic scale and thus not a linear scale. Additionally, there may be reasons to increase or decrease the range of acceptable pH levels or provide a specific single point pH level.

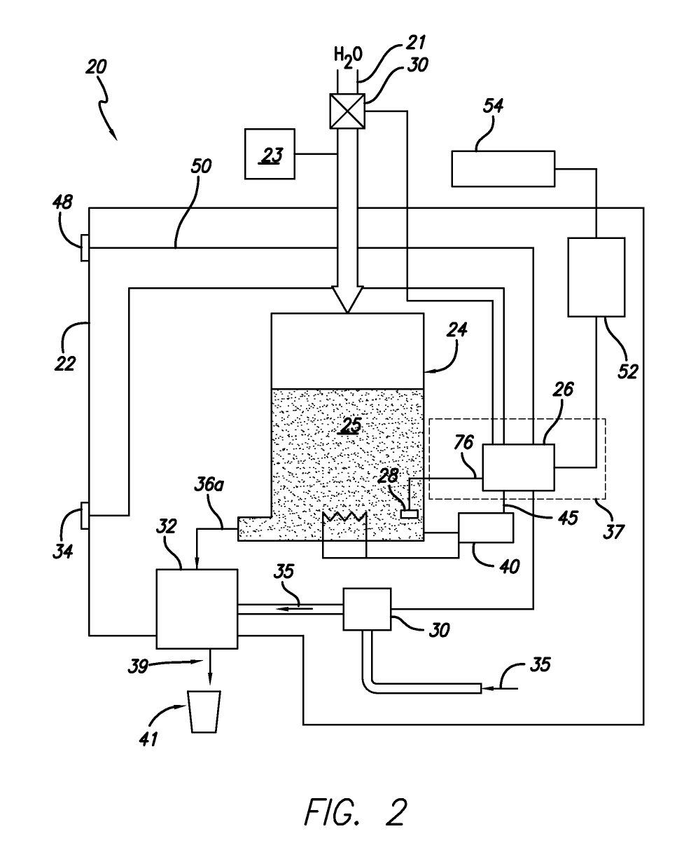

The present disclosure provides an apparatus, system and method including a pH sensor assembly for use with or in combination with a beverage container. Referring to FIG. 2, one embodiment of a beverage dispenser 20 according to the present disclosure includes a housing 22 which contains a container 24, a controller 26, a pH sensor assembly 28, a solenoid valve 30, mixing assembly or apparatus 32, and switch 34. The pH sensor assembly 28 and controller 26 define an apparatus for detecting a pH or acidity level of a beverage material 25 in the container 24.

The beverage material 25 may be prepared by combination of a liquid, such as from hot water inlet 21 and a beverage powder or mix from a beverage preparation hopper 23. Beverage powder and mix as used here as exemplary and one skilled in the art will appreciate that many known methods for preparing a beverage may be used, such as brewing a coffee or tea beverage from ground beans or leaves. Preferably, the beverage material comprises a brewed or mixed beverage made from coffee or tea products, which may include liquid extracts.

Container 24 is made from a material which is generally capable of containing a beverage 25 and holding a beverage for extended periods of time. Such a hopper material might include plastics, glass or metals which might suitably perform the desired characteristics of a container. The pH sensor assembly 28 senses the acidity of the beverage 25 and generates a signal communicated to and received by the controller 26. The controller 26 compares the signal to a pre-determined pH level or change in pH, which may be assigned to a particular beverage. The controller 26 may operate heating element 38, may display an indication of the suitability of non-suitability of the beverage via sensor device 48, or may restrict operation of solenoid valve 30, mixing assembly or apparatus 32, and/or switch 34 to restrict further dispensing of beverage 25. Sensor assembly 28 transmits a signal to controller 26 in relation to the pH sensed. Controller 26 receives the signal and may provide an output, such as power, to the display 48 and an output signal 50 to control the operation of display 48 and other devices, such as restricting, ceasing, locking out or otherwise controlling operation of solenoid valve 30 when the pH level in container 24 is below a desired level or otherwise meets pre-determined criteria.

Terms including beverage, brewed, brewing, brewing substance, brewed liquid, and brewed beverage as may be used herein are intended to be broadly defined as including, but not limited to, the brewing of coffee, tea and any other beverages. This broad interpretation is also intended to include, but is not limited to any process of dispensing, infusing, steeping, reconstituting, diluting, dissolving, saturating or passing a liquid through or otherwise mixing or combining a beverage substance with a liquid such as water without limitation to the temperature of such liquid unless specified. The disclosed system, method or apparatus will work with any type of food or beverage regardless of the temperature. This broad interpretation is also intended to including, but is not limited to beverage substances such as ground coffee, tea, liquid beverage concentrate, powdered beverage concentrate, flaked, granular, freeze dried or other forms of materials including liquid, gel, crystal or other forms of beverage or food materials to obtain a desired beverage or other food product.

One of the benefits of the disclosed system, method and apparatus is that it reliably senses the degradation level or “freshness” independent of time, optical properties, temperature or other characteristics, each of which individually or in combination may affect the “freshness” of the beverage. Additionally, since there is a wide range of physical characteristics of the beverage such as powdered, flaked, granular, freeze dried or other forms of materials including liquid, gel, crystal or other forms of beverage or food materials, the disclosed system, method and apparatus accommodates this wide range of physical characteristics and reliably detects the freshness of the beverage in the container. Time and opacity determination being susceptible to inaccurate determinations because a primary character or indicator of freshness or undesirable beverage is not being detected.

In general, the system operates when switch 34 is activated to initiate a dispensing cycle in which water 35 controllably passing through solenoid valve 30 is mixed with beverage 25 in mixing assembly 32. The dispenser 20 dispenses a beverage 39 to a corresponding cup or container 41. Controller 26 operates solenoid valve 30 associated with a water distribution system to combine beverage 25 and water in mixing assembly 32 and dispense a resultant beverage therefrom. Sensor assembly 28 is coupled to controller 26 for detecting the pH level of beverage 25 in container 24. It is envisioned that the disclosed apparatus, system and method of pH level detection will also operate with other embodiments of dispensers. The container 24 retains a volume of beverage material 25.

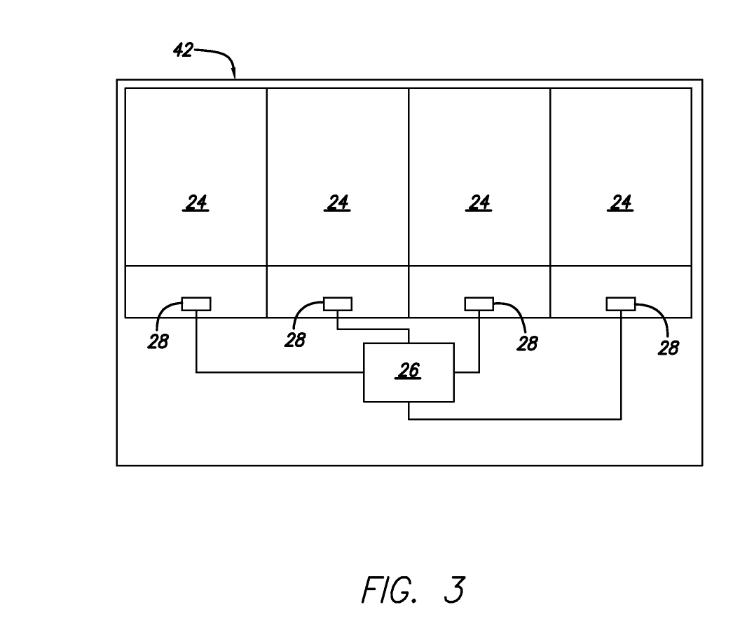

FIG. 3 shows a multiple server beverage dispenser in accordance with the present disclosure. Using sensor assemblies 28 positioned at the rear of each server 24, a series of servers 24 can be positioned within a single housing 42 allowing close side by side orientation of multiple servers 24 within housing 42. A single controller 26 may be coupled to all of the multiple sensors 28. As this is a rear side view dispensary faucets or nozzles are provided on the opposite side and thus not shown in FIG. 3.

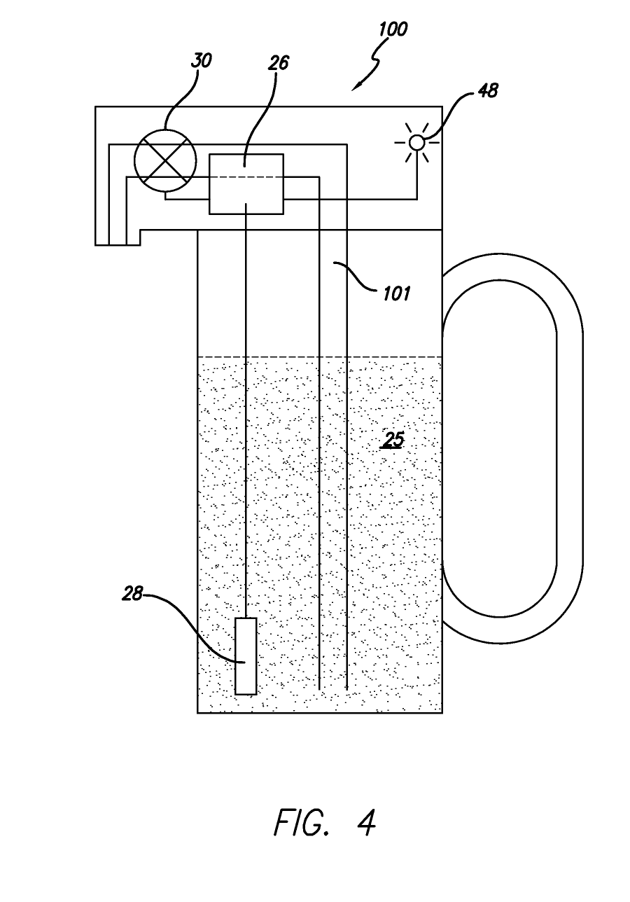

Referring now to FIG. 4, pH sensor assembly 28 detects the pH level in container or beverage server 100. The sensor assembly 28 is positioned to communicate with the interior of server 100 near the bottom of server 100 so that pH levels may be sensed during low volume situations. Sensor assembly 28 provides a signal to controller 26, which may operate and a sensory device or display 48 or may disable solenoid valve 30 to restrict the ability to dispense beverage 25 through dispensing tube 101. The operation of display 48 provides information to a user that a pre-determined unacceptable pH value has been reached, so that fresh beverage may be prepared.

In a further embodiment (not shown), the sensor assembly 28 is positioned adjacent or near the bottom of the container, and electrical leads extend through the container wall and terminate in electrical pads adapted to communicate with cooperative electrical pads on a warming plate to provide for communication of power to the pH sensor assembly and communication of the sensing signal from the assembly to a controller. As such, the sensor assembly may include a rechargeable battery to operate an indicator light operable from signals generated intermittently by the pH sensor.

A method of monitoring the freshness of a beverage using a pH level sensor includes preparing a beverage and retaining the beverage in a container for serving, sensing the pH level of the beverage, providing an indication of freshness based on the pH level of the beverage being within or below of a defined parameter or range of parameters. A further method of monitoring the freshness of a beverage using a pH level sensor includes preparing a beverage and holding the beverage in a container, sensing the pH level of the beverage, and restricting delivery of a beverage when the pH level is beyond or outside of a defined parameter or range of parameters such as when the pH level is low. The delivery of a beverage may be restricted, ceased or otherwise controlled by controlling the flow of beverage from the container.

As indicated above, when the sensor assembly 28 indicates the pH has dropped to a sufficiently low level or changed over a sufficiently large range, it will transmit a signal 76 to the controller 26. The signal 76 received by the controller 26 can be used to activate an indicator light 48 or other indicia such as an auditory signal. Also, the controller 26 can send a signal to a transmitter 52 which can transmit the information by RF or other communication means to a monitor 54.

A pH level condition can also be used by the controller 26 to lock out operation of the solenoid valves 30 and/or the switch 34. When a pH level is detected the indicator light 48 will be activated and the switch and/or solenoid valves 30 will be deactivated. This prevents continued dispensing from the apparatus 20 when the pH level is insufficient to produce a quality beverage product.

While this disclosure has been described as having an exemplary embodiment, this application is intended to cover any variations, uses, or adaptations using its general principles. It is envisioned that those skilled in the art may devise various modifications and equivalents without departing from the spirit and scope of the disclosure as recited in the following claims. Further, this application is intended to cover such departures from the present disclosure as come within the known or customary practice within the art to which it pertains.

1. A beverage container including a freshness detecting system for detecting the freshness of the beverage retained in the container, comprising:
a pH sensor associated with the container for detecting the pH of beverage retained in the container;
a controller coupled with the pH sensor for receiving a signal from the sensor; and
an output device coupled to the controller for using information from the controller relating to the pH level sensed by the pH sensor.

2. The beverage container including a freshness detecting system for detecting the freshness of the beverage retained in the container of claim 1, in which the output device is an indicator capable of providing a sensory signal based on the pH level sensed by the pH sensor.

3. The beverage container including a freshness detecting system for detecting the freshness of the beverage retained in the container of claim 1, in which the output device is a controllable valve associated with the container and is controllable based on the pH level sensed by the pH sensor.

4. The beverage container including a freshness detecting system for detecting the freshness of the beverage retained in the container of claim 1, in which the pH sensor is positioned in the container contacting at least a portion of beverage retained in the container for detecting the pH of the beverage.

5. The beverage container including a freshness detecting system for detecting the freshness of the beverage retained in the container of claim 1, in which the sensor is coupled to the controller by means of at least one of conductive connection, optical connection, and wireless connection.

6. The beverage container including a freshness detecting system for detecting the freshness of the beverage retained in the container of claim 1, in which the sensor is carried on a lid assembly associated with the container.

7. The beverage container including a freshness detecting system for detecting the freshness of the beverage retained in the container of claim 1, in which the sensor is carried on a liquid level detecting assembly.

8. The beverage container including a freshness detecting system for detecting the freshness of the beverage retained in the container of claim 1, in which an indicator is coupled to the controller, the indicator providing a signal to a user of the state of the freshness of the beverage as detected by the pH sensor.

9. The beverage container including a freshness detecting system for detecting the freshness of the beverage retained in the container of claim 1, in which the controller controls a function of at least one of the container and associated system in response to the signal from the pH sensor.

10. The beverage container including a freshness detecting system for detecting the freshness of the beverage retained in the container of claim 1, in which the controller controls a dispense valve in response to the pH level being outside of a predetermined range.

11. A beverage system including a container including a freshness detecting system for detecting the freshness of the beverage retained in the container, comprising:
a pH sensor associated with the container for detecting the pH of beverage retained in the container;
a controller coupled with the pH sensor for receiving a signal from the sensor; and
an output device coupled to the controller for using information from the controller relating to the pH level sensed by the pH sensor.

12. The beverage system including a container including a freshness detecting system for detecting the freshness of the beverage retained in the container of claim 11, in which the output device is an indicator capable of providing a sensory signal based on the pH level sensed by the pH sensor.

13. The beverage system including a container including a freshness detecting system for detecting the freshness of the beverage retained in the container of claim 11, in which the output device is a controllable valve associated with the container and is controllable based on the pH level sensed by the pH sensor.

14. The beverage system including a container including a freshness detecting system for detecting the freshness of the beverage retained in the container of claim 11, in which the pH sensor is positioned in the container contacting at least a portion of beverage retained in the container for detecting the pH of the beverage.

15. The beverage container including a freshness detecting system for detecting the freshness of the beverage retained in the container of claim 11, in which the sensor is coupled to the controller by means of at least one of conductive connection, optical connection, and wireless connection.

16. The beverage container including a freshness detecting system for detecting the freshness of the beverage retained in the container of claim 11, in which the sensor is carried on a lid assembly associated with the container.

17. The beverage container including a freshness detecting system for detecting the freshness of the beverage retained in the container of claim 11, in which the sensor is carried on a liquid level detecting assembly.

18. The beverage container including a freshness detecting system for detecting the freshness of the beverage retained in the container of claim 11, in which an indicator is coupled to the controller, the indicator providing a signal to a user of the state of the freshness of the beverage as detected by the pH sensor.

19. The beverage container including a freshness detecting system for detecting the freshness of the beverage retained in the container of claim 11, in which the controller controls a function of at least one of the container and associated system in response to the signal from the pH sensor.

20. The beverage container including a freshness detecting system for detecting the freshness of the beverage retained in the container of claim 11, in which the controller controls a dispense valve in response to the pH level being outside of a predetermined r

21. A method of monitoring beverage freshness of a beverage dispensed into a beverage container, the method comprising:
providing a pH sensor associated with the container;
detecting the pH of beverage retained in the container;
providing a controller coupled with the pH sensor;
communicating a signal from the sensor to the controller
providing an output device coupled to the controller
communicating information from the controller to the output device relating to the pH level sensed by the pH sensor.
